(19) United States
(12) Patent Application Publication
(10) Pub. No.: US 2023/0234918 A1
(43) Pub. Date: Jul. 27, 2023

(54) AUTOMATED DIAZOMETHANE GENERATOR, REACTOR AND SOLID PHASE QUENCHER

(71) Applicant: COUNCIL OF SCIENTIFIC AND INDUSTRIAL RESEARCH, New Delhi (IN)

(72) Inventors: Ajay Kumar SINGH, Tarnaka, Hyderabad (IN); Srihari PABBARAJA, Tarnaka, Hyderabad (IN); Deepak Kumar JAISWAL, Tarnaka, Hyderabad (IN); Dnyaneshwar AAND, Tarnaka, Hyderabad (IN); Abhilash RANA, Tarnaka, Hyderabad (IN)

(21) Appl. No.: 18/042,169

(22) PCT Filed: Aug. 24, 2021
(86) PCT No.: PCT/IN2021/050811

§ 371 Date: Feb. 17, 2023

(30) Foreign Application Priority Data
Aug. 24, 2020 (IN) 202011036463

(51) Int. Cl. C07C 245/16; B01J 19/24; B01J 19/00

(52) U.S. Cl. CPC C07C 245/16; B01J 19/0093; B01J 19/2415; B01J 2219/00905

(57) ABSTRACT

A process for producing diazomethane of Formula 1 (CH₂N₂), with an automated apparatus is described. A stock solution of N-methyl-N-nitroso amine in an organic solvent is continuously flown and mixed with an aqueous inorganic base at a T-mixer to form a mixture. Then it is passed through a capillary micro reactor at a temperature in a range of 20 to 30° C. to form diazomethane. The mixture is separated into an aqueous layer and an organic layer using a continuous flow micro-separator. The organic layer has 0.1-0.4 M diazomethane. The organic layer is reacted with a carboxylic acid, phenol, an alkyne, an anhydride, a carboxyl metal organic framework (MOF), or MOF coated cotton to form a corresponding ester, a pyrazole, an ether, a diazo ketone, a stable carboxyl MOF or a stable MOF coated cotton fiber.

### CROSS REFERENCE TO RELATED APPLICATIONS

This application is a National Stage application of PCT/IN2021/050811, filed Aug. 24, 2021, which claims priority to India Application No. 202011036463, filed Aug. 24, 2020, and all the benefits accruing therefrom under 35 U.S.C. § 119, the content of which is incorporated by reference in its entirety.

### TECHNICAL FIELD

The disclosure describes a diazomethane generator and a process for producing diazomethane and more specifically describes an automated diazomethane generator, reactor, and a solid phase quencher and a process for producing and quenching diazomethane with the automated generator.

### BACKGROUND

Diazomethane has a wide range of utilities for introducing methyl or methylene group in carboxylic acids, phenols, alcohols, enols, and heteroatoms, also used for the ketones ring expansion or chain extension, and ketones to epoxides conversion, etc. Furthermore, an example of its use is in the conversion of acid chlorides to α-diazoketones, cycloaddition reactions with olefins to produce cyclopropyl or nitrogen-containing heterocyclic rings, homologation of ketones or amino acids. Further examples involve the multi-step syntheses of drugs and natural products, including bactericides, pesticides, functional chemicals, solvents for polymerization reactions, and the formation of API intermediates such as Saquinavir (Roche Laboratories), sitagliptin, etc. One of the extraordinary performances of diazomethane is the addition of carbon must be done without compromising the chirality of the substrate or affecting any remained portion of the molecule. Especially for Arndt-Eistert reaction, no other alternative reagents are available, and diazomethane becomes necessary. Despite its wide synthetic versatility, diazomethane is a highly dangerous reagent because of its carcinogenic, allergen, poisonous and explosiveness nature.

In the batch process synthesis of diazomethane caution has to be taken against the use of ground-glass joints and any glassware that has not been fire polished. In past decades, a series of specifically designed equipment for diazomethane preparation, such as the apparatus of Aldrich Chemical Company, Inc., Milwaukee, Wis., USA, Aldrichimica Acta 16(1): 3-10 (1983), Erlenmeyer glassware etc. are available. The point should be noted here that for making the anhydrous diazomethane, classic distillation techniques have been applied. The highly explosive and genotoxic nature of diazomethane is its reaction with nucleophilic DNA, wherein even a few ppm exposures cause a sore throat with fever and difficulty in breathing. As a result, workers in this field have to face serious safety issues in the generation, distillation, and transportation, because of which many potential opportunities are turned away. Thus, there is a need to develop a safe and efficient chemical approach to expand the scope of diazomethane chemistry, to new unexplored dimensions.

To avoid the distillation process, several lab-scale continuous-flow tools have emerged for the safe and convenient in situ on-demand production of potentially toxic, reactive, or explosive intermediates. It has now been discovered that diazomethane can be synthesized in a flow reaction on a small scale with little or no danger of explosion. However, the unavailability of the modular flow chemistry equipment’s and the high price involved in designing the reactor, untrained manpower, lab-scale productivity, embedded membrane low permeability, fouling, low flexibility etc. limits the industrial applicability. On other hand, a high stoichiometric amount of diazomethane is needed for the chemical reaction leading to the exposure of unused diazomethane post-synthetic work-up process. The quenching process for the excess unused diazomethane has not been explored.

To realize the above-mentioned problem, an automated diazo-pen for the laboratory scale and a diazo-cube for the kilo-scale is described and the same can be extended as parallelized diazo-cube for the industrial scale.

In view of the existing limitations, the present disclosure provides an automated diazo-pen system for multi-operations, without any intermediate purification, solvent exchange, and synthesis of active pharmaceutical ingredients (API).

The disclosure further provides an industrial scale diazo-cube system that can carry out a multi-step process system in a completely safe manner.

### SUMMARY

Aiming at the defects and limitations, a new and automated multi-operational continuous flow reactor system (diazo-pen/diazo-cube) for the preparation of diazomethane and analogs thereof is developed.

In an embodiment, the present disclosure provides, an ultra-fast continuous flow reactor system for preparation of diazomethane thereof of formulalthrough diazo-pen or diazo-cube:

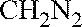

(1) In a first embodiment, diazomethane intermediate and its further product thereof may be prepared by formula2 reacting with a base of formula3, extracting with an organic solvent, and aq. org. separation through the micro-separator to get the compound formula1;

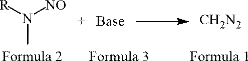

(2) In a second embodiment, on-demand synthesized diazomethane through the diazo-pen was utilized in the following reactions:

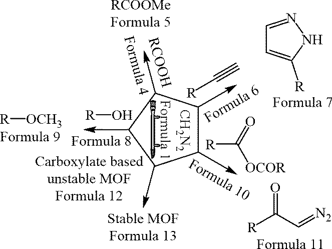

(a)Diazo-pen for esterification of formula4 by reacting with formulal to obtain a compound of formula5.

(b) Diazo-pen for pyrazole ring formation of formula6 by reacting with formula1 to obtain a compound of formula7.

(c) Diazo-pen for phenolic group protection of formula8 by reacting with formula1 to obtain a compound of formula9.

(d) Diazo-pen for conversion of formula10 by treating with formula1 to obtain a compound of formula11.

(e) Diazo-pen for stabilization of the carboxylated MOF of formula12 by reacting with formula1 to obtain a compound of formula13.

In a third embodiment, industrial scale in-situ diazomethane generation, extraction, separation and further consumption through the reagent and finally passing through the newly developed quencher for the degradation of unused diazomethane.

According to the first embodiment, may be performed in the presence of anamine compound formula2. Examples of suitable formula include N-methyl-N′-nitro-N-nitrosoguanidine, N-methyl-N-nitrosourea (NMU), N-methyl-N-nitrosocarbamate, N-methyl-N-nitrosourethane, and N-methyl-N-nitroso-p-toluenesulfonamide and mixtures thereof.

According to first embodiment, may be performed in the presence of a base compound of formula3. Examples of suitable formula include KOH, NaOH, NH₄OH, LiOH, RbOH, CsOH, Ca(OH)₂, Ba(OH)₂, Sr(OH)₂, and mixtures thereof.

According to the first embodiment, may be performed in the presence of an organic solvent. Examples of suitable formula include methanol, ethanol, isopropanol, THF, diethyl ether, dimethyl ether, toluene, MTBE, acetonitrile, dichloromethane, dichloroethane, tetrahydrofuran, ethyl acetate, isopropyl acetate, dimethylformamide, dimethyl sulfoxide, acetone, N-methyl pyrrolidone, and mixtures thereof.

According to the first embodiment, may be performed in the capillary microreactor. Examples of suitable micro-reactor include PTFE, PFA, PE, SS-316, haste alloy, glass, and mixtures thereof.

According to the first embodiment, aq. org continuous separation may be performed with membrane separator, density-based separation, hydrophobicity-based separation, filter paper, and mixtures thereof.

According to the second embodiment step (a), may be performed in the presence of a carboxylic acid compound formula4. Examples of suitable formula4 include benzoic acid, 4-nitrobenzoic acide, 4-ethoxybenzoic acid, 3,5-dimethylbenzoic acid, 4-(benzyloxy) benzoic acid, and 3-bromo-4-methyl benzoic acid and mixtures thereof.

According to the second embodiment step (b), may be performed in the presence of an alkyne compound formula6. Examples of suitable formula6 include 1-ethynyl-4-methylbenzene, and mixtures thereof.

According to the second embodiment step (c), may be performed in the presence of a phenol compound formula8. Examples of suitable formula8 include 4-bromo phenol, 4-bromo-2-methoxyphenol, and mixtures thereof.

According to the second embodiment step (d), may be performed in the presence of anhydride (R)- compound formula10. Examples of suitable formula10 include 2-(((benzyloxy)carbonyl)amino)-3-phenylpropanoic (ethyl carbonic) anhydride, and mixtures thereof.

According to the second embodiment step (e), may be performed in the presence of carboxylated MOF formula12. Examples of suitable formula12 includeHKUST, HKUST-coated cotton fiber, UiO-66, MIL-100, Eu-MOF, MIL-101-(Cr), MIL-101-(Cr), and mixtures thereof.

According to the third embodiment (diazo-cube), may be performed in the presence of carboxylic acid, alkyne, alcohol, and carboxylated MOF.

### BRIEF DESCRIPTION OF THE DRAWINGS

FIG. 1: Flow diagram of the integrated continuous-flow process system for the diazomethane generation (1);

FIG. 2: Schematic representation for the utilization of diazo-pen in various reactions;

FIG. 3: ATR-IR analysis of the pristine HKUST and varied time diazomethane treated HKUST;

FIG. 4: SEM and EDX analysis image of the pristine HKUST and one hour diazomethane treated HKUST-60 M;

FIG. 5: Powder XRD analysis of the pristine HKUST and one-hour diazomethane treated HKUST;

FIG. 6: Hydrophobicity analysis of the pristine HKUST and one-hour diazomethane treated HKUST;

FIG. 7: Color change experiment of the HKUST-coated cotton and one-hour diazomethane exposed HKUST;

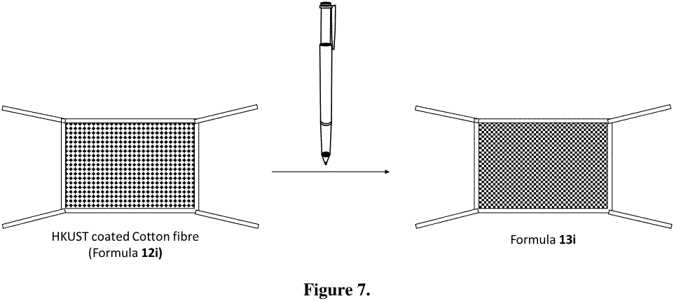

FIG. 8: SEM and EDX analysis image of the pristine UiO-66 and one hour diazomethane treated UiO-66-60 M;

FIG. 9: ATR-IR analysis of the pristine UiO-66 and varied time diazomethane treated UiO-66-60M;

FIG. 10: ATR-IR analysis of the pristine MIL-101-Cr and varied time diazomethane treated MIL-101-Cr-60 M; and

FIG. 11: Schematic presentation of the diazo-cube.

### LIST OF ABBREVIATIONS

BPR = Back pressure regulator
DCE = Dichloroethane
MeCN = Acetonitrile
TEA = Triethylamine
MTBE = Methyl tert-butyl ether
THF = Tetrahydrofuran
MeOH = Methanol
DEE = Diethylether
KOH = Potassium Hydroxide
ETFE = Ethylene tetrafluoroethylene
HPLC = High pressure Liquid chromatography
HRMS = High resolution mass spectroscopy
ID = Inner Diameter
IR = Infra-red
XRD = X-ray diffraction
SEM = Scanning Electron Microscope
NMR = Nuclear Magnetic resonance
OD = Outer Diameter
PE = Polyethylene
PFA = Perfluoroalkoxy alkane
PTFE = Polytetrafluoroethylene
SS = Stainless Steel
TLC = Thin layer chromatography
UV = Ultra-Violet
API = Active Pharmaceutical Ingredient
MOF = Metal-Organic Framework
HKUST = Combination of Copper (II)and benzene-1,3,5-tricarboxylate ligand-based MOF
MIL-100 (Al) = Combination of Aluminum(III) and benzene-1,3,5-tricarboxylateligand-based
MOF, MIL 101(Cr) =Combination ofChromium (III) and terephthalateligand-based MOF, MIL
101(Fe) =Combination ofIron (III) and terephthalate ligand-based MOF, UiO-66 =Combination of Zirconium(IV) and terephthalate ligand-based MOF.

### DETAILED DESCRIPTION

As used herein, the modifier “about” should be considered as disclosing the range defined by the absolute values of the two endpoints. For example, the expression “from about 1 to about 4” also discloses the range “from 1 to 4.” When used to modify a single number, the term “about” may refer to ±10% of the said number including the indicated number. For example, “about 10%” may cover a range of 9% to 11%, and “about 1” means from 0.9-1.1.

As used herein, the term “reduced pressure” refers to a pressure that is less than atmospheric pressure. For example, the reduced pressure is about 10 mbar to about 50 mbar.

As used herein, the term “pump” refers to a device that moves fluids (liquids or gases), or sometimes slurries, by mechanical action.

As used herein, the term “protic solvents” refers to any organic solvent that contains a labile H⁺ and vice-versa for the aprotic solvent.

As used herein, the term “protic acid” refers to any reagent that contains a labile H⁺ and vice-versa for the product.

As used herein, the term “base” refers to any reagent that contains a labile OH⁻ or proton acceptor and vice-versa for the product.

In the first embodiment, the present disclosure provides, a continuous flow process system for the highly safe automated diazomethane generator (Pen and Cube) thereof of formulal

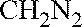

In another embodiment, the present disclosure provides a process using an integrated continuous flow reactor system diazo-pen for the preparation of diazomethane of formula1, extraction, and membrane-based liquid-liquid separation.

In yet another embodiment, the diazo-pen comprise of a syringe pumps, tubular micro-reactor, and micro-separator consisting of the long-serpentine tunnel sandwiched in a PTFE-hydrophobic membrane with three alternate polytetrafluoroethylene (PTFE) sheets with the identical dimension of groove channels sandwiched between two metal holders tightly pressed by the screw to seal the device for prevention of leaks.

In a preferred embodiment, the middle part membrane micro-separator comprises of an assembly of specially designed laser grooved micro-patterned PTFE sheetwith hydrophobic PTFE membrane; wherein, the hydrophobic membranehas an average pore size 0.25-0.45 mm.

In one embodiment, the present disclosure provides a highly safe process for the synthesis of utilization of diazomethane comprising the steps of:

### In Diazomethane Generation Step

introducing a solution of NMU of formula2 and a base of formula3 in a mixture of solvent to the diazo-pen reactor and maintaining the reaction mixture in the reactor for about 1-10 min. at a temperature of range 0-40° C. and at a pressure of about 0-5 bar to obtain compounds of formula1.

In one embodiment, the solvent for the reaction in step a) is a mixture of solvents selected from the group of: methanol, ethanol, isopropanol, THF, diethyl ether, dimethyl ether, toluene, MTBE, acetonitrile, dichloromethane, dichloroethane, tetrahydrofuran, ethyl acetate, isopropyl acetate, dimethylformamide, dimethyl sulfoxide, acetone, N-methyl pyrrolidone, and mixtures thereof.

Table 1 represents the optimization of the model reaction of step a) with a diazo-pen reactor and in general, reaction performance was found to be dependent on the flow rate (residence time), solvent, and temperature. After studying several reaction conditions, finally, 86 % yield of diazomethane (2.5 mmol h⁻¹ productivity Table 1, entry 3) was obtained in 4.5 min residence time and at ambient temperature.

Table 1

Optimization of formula1 synthesis in the continuous flow process

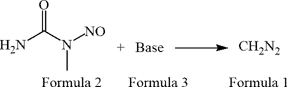

| Entry | Flow rate (µL/min) | | Retention time (min.) | | Yield (%) |
|---|---|---|---|---|---|
| | formula2 | formula3 | Reaction | Separation | |
| 1 | 100 | 100 | 10 | 3.5 | 68 |
| 2 | 200 | 200 | 5 | 1.7 | 68 |
| 3 | 300 | 300 | 3.3 | 1.2 | 86 |
| 4 | 400 | 400 | 2.5 | 0.9 | 78 |
| 5 | 500 | 500 | 2 | 0.7 | 62 |
| 6 | 600 | 600 | 1.6 | 0.6 | 72 |

Reaction condition: formula2 = 0.162 M in MeOH &diethyl ether in 1:2 ratios, formula3= 30 wt% in water; Formula1 yields are based on benzoic acid titration.

When results were compared with previously reported literature in a conventional batch process/flow process, it’s worth mentioning here that the batch process needs a high temperature (45-60° C.), areaction time of 3 h, further extraction time of 0.5-1 h and then distillation at a higher temperature, with unnecessary catalyst diethylene glycol monoethyl ether (U.S. Pat., 1998, USOO5817778A.

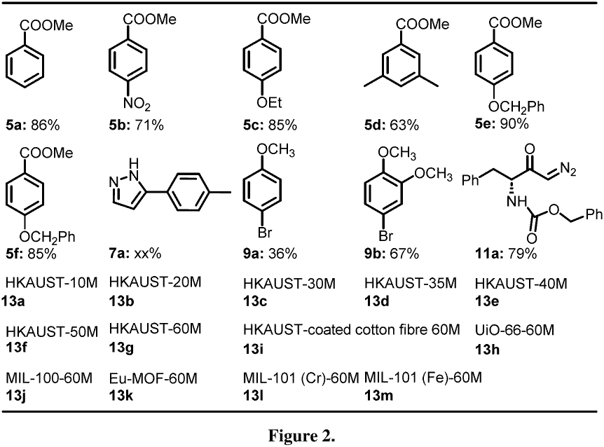

FIG. 2 is an illustration of a schematic integrated continuous flow total process system for the production of formula5-13. The diazo-pen total process system consists of the components viz. synthesis, quenching, extraction, liquid-liquid micro-separator to continuous generation of the diazomethane.

### (A) Diazo-Pen for the Ester Formation Reaction

The out-flowingcrude mixture of formula1 from diazo-penand separately formula4 are dissolved in a suitable aprotic solvent and stirred under batch process to get formula5. After studying several parameters, finally individual step set gave 63-90% yield of formula5 obtained in 21 min.diazomethane exposure (FIG. 2).

### (B) Diazo-Pen for the Pyrazole Synthesis Reaction

The out-flowingcrude mixture of formula1 from diazo-pen and separately the 1-ethynyl-4-methylbenzene dissolved in a suitable aprotic solvent are mixed together and stirred under batch process to get formula7. After studying several parameters, finally,the pyrazole step gave 41% yield of formula7in 21 min. diazomethane exposure (FIG. 2).

### (C) Diazo-Pen for the Phenolic Group Protection Reaction

The stock solution of substituted phenol was prepared in round bottomed flask and diazo-pen outletis directly connected with the RB and ensured that there is no leak in the system. The diazo-pen was set to infuse the instantly prepared diazomethane for phenolic group protection. In general phenolic group protection depends on the reaction time, temperature and pressure and finally 36-67% yield of formula9 was obtained in 21 min of the reaction time. After completion of the reaction, the reaction mixture was evaporated under reduced pressure to remove excess DEE. The resulting mixture was extracted through the known prior art. The organic phase was dried over anhydrous sodium sulfate, filtered, and concentrated.

### (D) Diazo-Pen for the Arndt-Eistert Synthesis Reaction

In general, freshly prepared (R)-2-(((benzyloxy)carbonyl)amino)-3-phenylpropanoic (ethyl carbonic) anhydride was dissolved in an aprotic solvent and stirred with instantly generated diazomethane through the diazo-pen for the short period of the time. Finally, 79% yield of formula11 was obtained in 21 min. of the reaction time. The resulting mixture was extracted and isolated through the known prior art.

### (E) Stabilization of MOF Through the Diazo-Pen

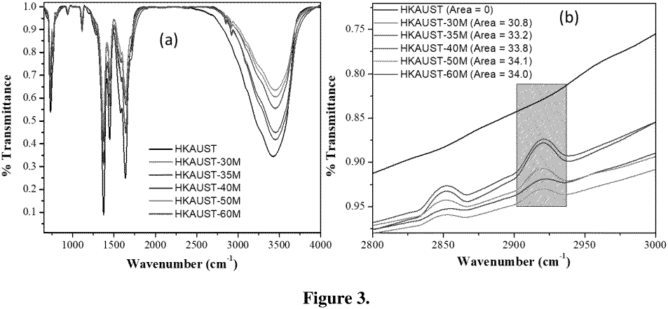

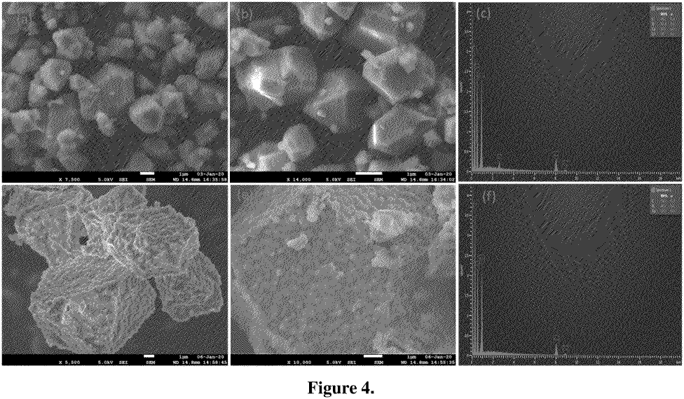

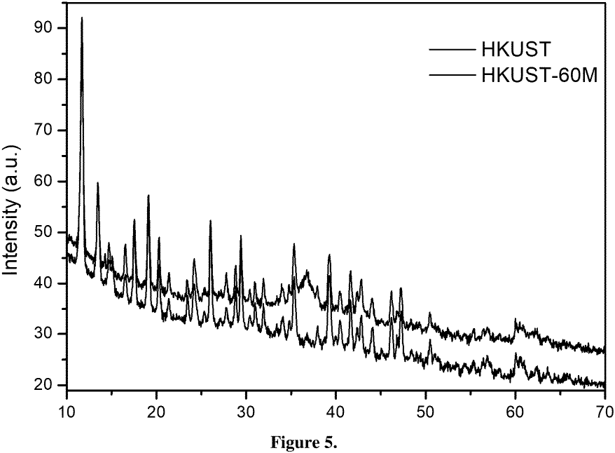

Carboxylate based metal-organic frameworks (MOFs) have ultra-high porosity and is mostly used in various applications such as gas storage, sensing, catalysis, and electroactive materials in devices. The basic problem with carboxylate-based MOFs is their instability in polar solvents. Cu based MOF (HKUST) has ultra-high porosity but due to the lack of their stability, several applications are unexplored. To solve the stability issue, we have chosen HKUST as the model MOF to treat with diazomethane. At first, we prepared the HKUST from the know prior art and directly exposed it with diazomethane generated from the diazo-pen for a limited time (0-60 Min.). Diazomethane is sensed through the color changing properties of the HKUST (dark blue to green) to get the formula13a-13g. In general, reaction performance is found to be dependent on the diazomethane exposure time and one hour time was found enough to saturate 100 mg of HKUST.To see the detailed insights in molecular level changing during the diazomethane exposure, we have conducted the ATR-IR analysis and two new peaks around the 2850 and 2925 cm⁻¹ appeared corresponding to the ester formation. The IR result shows that the unreacted carboxylic acid group is getting converted to ester form (FIG. 3). Further to see the topographical changes in terms of the shape and size of the MOF, we have conducted the SEM analysis and the results show that crystal size are maintained but the porosity has been increased (FIG. 4). Next, to check crystal defect during the diazomethane exposure, we conducted the powder XRD (FIG. 5)and the results show that there is no change in the crystal shape and size.

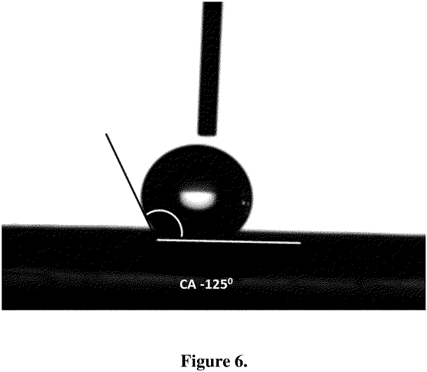

FIG. 6 describes the contact angle of the treated and untreated HKUST moiety and the result shows that the contact angle has been increased to 125 in formula13g. Further, we have checked the pH stability of the formula13g and the result shows that under pH 0-14 formula13g is stable.The next disclosure, to provide a wearable solid quencher for the diazomethane, HKUST MOF was coated over the cotton surface and exposed to the diazomethane gas.The color changedfrom blue to green indicating for the diazomethane absorption and degradation.

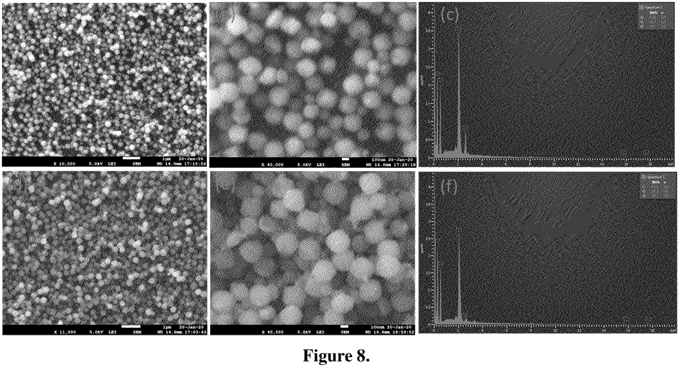

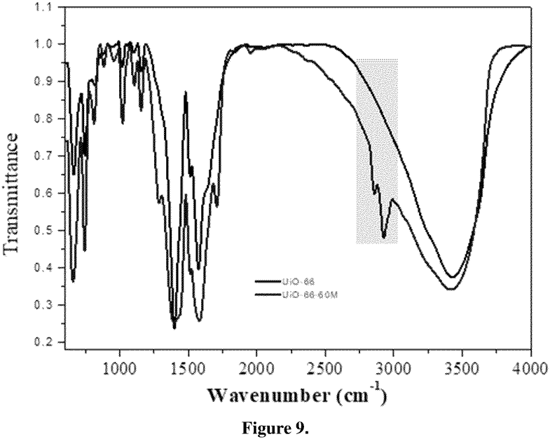

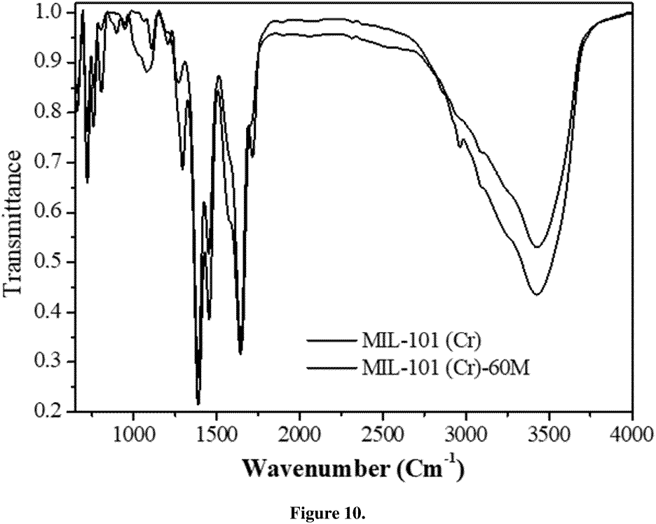

Next, we have synthesized several carboxylated based MOF (UiO-66, MIL-100, Eu-MOF, MIL-101 (Cr), MIL-101(Fe) through the know prior art and directly exposed with diazo-pen for one hour and further samples were characterized through the various analytical technique as shown in FIGS. 8-10.

### Diazo-Cube

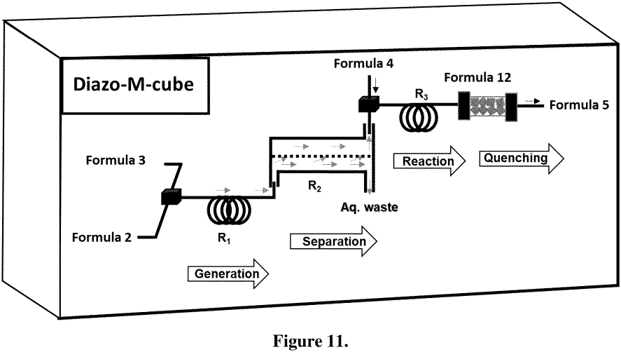

Diazo-pen is based on the automated syringe pump and for each and every experiment one needs to feed the formula2 and base solution formula3 and use them for the laboratory scale. Further to extend the disclosure, we have designed the diazo-cube platform (FIG. 11) consisting of microfluidic devices that enable in situ generation of the diazomethane reagent, its separation from the reaction products, subsequent synthesis of the desired product with the carcinogenic reagent and decomposition of the unreacted carcinogenic reagent by quenching, separating the final desired product, all in a safe sequential manner. In the diazo-cube, A solution of formula2 in MeOH:DEE and a solution of a base in water were introduced into the capillary microreactor with a T-mixer using pumps. The flow rate of the formula2 solution (0-30 ml/min) was kept at same the rate of the base solution (0-30 mL/min), in accordance with the stoichiometry of the reagent and substrates. The two solutions were introduced to a T-mixer in a flow rate with the ratio of (formula2: formula3) to maintain the stoichiometry, and then passed through a PTFE tubingfor the diazo-metane generation during 0-4 min of residence time and room temperature. After the successful completion, the aqueous and DEE continuous flow droplets were separated through our partially modified previously reported micro-separator. A residence time of 0-10 min, 0-10 bar pressure was found to be enough for the aqueous waste removal of the crude organic solution of formula1. Further, the out-flow solution from the micro-separator was connected with a recirculatory pump, and a solution of acid or phenol or alkyne or alkene or anhydride, or aldehyde wastaken in a bottle and connected with the pump as described in FIG. 11. The flow rate of the formulal solution was kept in accordance with the stoichiometry of reagent and substrates and smoothly passed through perfluoroalkoxy (PFA) tubing with short residence time and ambient temperature and pressure for the reaction to occur. Next, the excess diazomethane in the out-flowing reaction mixture was passed through the HKUST MOF filled catalyst cartridge. The post-synthetic process was performed as per the prior art.

### Material and Method Used in Experiments

Most of the reagents and chemicals are bought from Spectrochem or AVRA or Sigma-Aldrich, which were used as such without any further purification. Common organic chemicals and salts were purchased from AVRA chemicals, India.

Deionized water (18.2 mS conductivity) was used in all experiments. All work-up and purification procedures were carried out with reagent-grade solvents. Analytical thin-layer chromatography (TLC) was performed using analytical chromatography silica gel 60 F254 precoated plates (0.25 mm). The developed chromatogram was analysed by UV lamp (254 nm). PTFE (id = 100-1000 µm) tubing, T-junction, and back-pressure controller (BPR) were procured from Upchurch IDEX HEALTH & SCIENCE. The pumpwas purchased from KNAUER. SS318 capillary bought from the spectrum market, Mumbai, India. The heating reactor was bought from Thales Nano Nanotechnology, Inc.

### Measurement Method

High-resolution mass spectra (HRMS) were obtained from a JMS-T100TD instrument (DART) and Thermo Fisher Scientific Exactive (APCI).

Nuclear magnetic resonance (NMR) spectra were recorded on a Bruker 600, 500, 400 or 300 MHz in CDCl₃ or DMSO-d₆ solvent. Chemical shifts for ¹H NMR are expressed in parts per million (ppm) relative to tetramethyl silane (δ 0.00 ppm). Chemical shifts for ¹³C NMR are expressed in ppm relative to CDCl₃ (δ 77.0 ppm). Data are reported as follows: chemical shift, multiplicity (s = singlet, d = doublet, dd = doublet of doublets, t = triplet, q = quartet, quin = quintet, sext = sextet, m = multiplet), coupling constant (Hz), and integration. GC/MS analysis was conducted on Shimadzu technology GCMS-QP2010 instrument equipped with a HP-5 column (30 m × 0.25 mm, Hewlett-Packard) and inbuilt MS 5975C VL MSD system with triple axis detector. ATR analysis was conducted on the Portable FTIR spectrometer Bruker ALPHA.

### General Procedure for the Synthesis of Formula 1

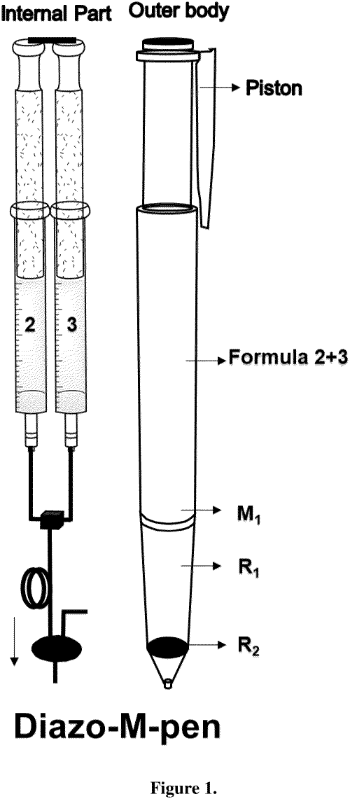

1. A solution of formula2 in MeOH and Diethyl ether, separately from a solution of formula3in water was taken in a syringe and connected with a pump as described in FIG. 1.

2. The flow rate of the formula2 solution was kept varied as the flow rate of formula3, in accordance with the stoichiometry of reagent and substrates, and smoothly passed through perfluoroalkoxy (PTFE) tubing (inner diameter (id) = 800-1000 µm, length = 1-4 m, volume =1.0-3.0 mL) for the reaction to occur.

3. A residence time of 1-10 min, 0-25° C., and pressure 0-1 bar was found to be enough for the diazomethane generation of formulal (Table 1).

4. Continuous-flow separation of the aqueous and organic layer was performed through our lab’s previously reported micro-separator. A residence time of 1-3 min, 0-1 bar pressure was found to be enough for the aqueous waste removal of the crude organic solution of formula1.

5. The generated formula1 was quenched and titrated with the carboxylic acid group.

### Example 1

### Diazo-Pen

A solution of formula2 in MeOH:DEE (1:2 ratio, 0.162 M) and a solution of KOH in water (30 wt%) were introduced into the capillary microreactor with a T-mixer using syringe pumps. The flow rate of the formula2 solution was kept at same the rate of the KOH solution, in accordance with the stoichiometry of the reagent and substrates. The two solutions were introduced to a T-mixer in a flow rate with the ratio of 1:33(formula2: formula3) to maintain the stoichiometry, and then passed through a PTFE tubing (id = 1000 µm, 1= 2.55 m, vol. = 2 ml) for the diazomethane generation during 3.3 min of residence time and room temperature (Table 1, entry 6). After the successful completion, the aqueous and DEE continuous flow droplet were separated through our partially modified micro-separator (Organic Process Research & Development, 2019, 23(9), 1892-1899). A residence time of 1.16 min, 0-1 bar pressure was found to be enough for the aqueous waste removal of the crude organic solution of formula1. The outflowed DEE reaction mixture was titrated with benzoic acid to get methyl benzoateconfirming theconcentration of diazomethane (0.21 M in DEE).

### General Procedure for the Synthesis of Formula 5

1. To an oven-dried 10-50 mL test tube equipped with a teflon coated magnetic stir bar, the carboxylic acid (1 mmol) was added. Then DEE or methanol or ethanol or THF (0-10 ml) were added using a syringe and further, the tube was sealed with septa, and an additional nitrogen balloon was placed over the tube.

2. Next, the diazomethane solution was added through the above designed diazo-pen for 0-21 min. (equivalent to 1 mmol of diazomethane).

3. After diazo exposure for 0-21 min, the product was washed with aq. NaHCO₃ (3×20 mL), then washed with brine (30 mL).

4. The organic phase was dried over Na₂SO₄ and concentrated under reduced pressure to provide a formula5.

### Example 2

Synthesis of methyl benzoate (5a):

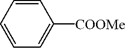

To an oven-dried 50 mL test tube equipped with a teflon coated magnetic stir bar, benzoic acid (122 mg, 1 mmol) was added. Then DEE (10 ml) was added using a syringe. Then the tube was sealed by septa and an additional nitrogen balloon was placed over the tube. Next, the diazomethane solution was added through the above designed diazo-pen for 21 min. (equivalent to 1 mmol of diazomethane). After diazo exposure for 21 min, the resulting product was washed with aq. NaHCO₃ (3×20 mL), then washed with brine (30 mL). The organic phase was dried over Na₂SO₄ and concentrated under reduced pressure to provide 5a as a colorless liquid (117 mg, 86%).

### Example 3

Synthesis of methyl 4-nitrobenzoate (5b):

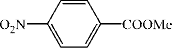 [SMILES: COC(=O)c1ccc([N+](=O)[O-])cc1]

The compound of formula (5b) was synthesised following the procedure described above under Example 2 and the general procedure involving corresponding reactants. The crude material was dried under reduced pressure to provide 5b as a white solid (128 mg, 71%); The spectra data matched with values reported in the literature (Tetrahedron Letters 2015, 56, 7008).

¹H NMR (400 MHz, CDCl₃) δ 7.72 (s, 1H), 7.16 (dd, J = 30.7, 7.8 Hz, 2H), 3.88 (s, 3H), 2.54 (s, 3H), 2.34 (s, 3H).

¹³C NMR (101 MHz, CDCl₃) δ 168.26, 137.03, 135.23, 132.74, 131.61, 131.02, 129.33, 51.77, 21.25, 20.80.

MS (EI): m/z 181.04 (M⁺).

### Example 4

Synthesis of methyl 4-ethoxybenzoate (5c):

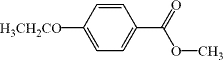

Compound of formula (5b) was synthesised following the procedure described above under Example 2 and the general procedure involving corresponding reactants. The crude material was dried under reduced pressure to provide 5c as a colorless liquid (153 mg, 85%); The spectra data matched with values reported in the literature (Organic Letters 2015, 17, 5276).

¹HNMR (400 MHz, CDCl₃) δ 7.97 (d, J= 9.0 Hz, 2H), 6.89 (d, J= 9.0 Hz, 2H), 4.07 (q, J= 7.0 Hz, 2H), 3.87 (s, 3H), 1.42 (t, J= 7.0 Hz, 3H).

¹³CNMR (101 MHz, CDCl₃) δ 165.85, 161.73, 130.54, 121.35, 113.01, 62.64, 50.78, 13.65.

MS (EI): m/z 180.08.

### Example 5

Synthesis of methyl 3,5-dimethylbenzoate (5d):

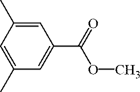

The compound of formula (5d) was synthesised following the procedure described above under Example 2 and the general procedure involving corresponding reactants. The crude material was dried under reduced pressure to provide 5d as a colorless liquid (153 mg, 85%); The spectra data matched with values reported in the literature (xxx).

¹H NMR (400 MHz, CDCl₃) δ 7.72 (s, 1H), 7.16 (dd, J= 30.7, 7.8 Hz, 2H), 3.88 (s, 3H), 2.54 (s, 3H), 2.34 (s, 3H).

¹³C NMR (101 MHz, CDCl₃) δ 168.26, 137.03, 135.23, 132.74, 131.61, 131.02, 129.33, 51.77, 21.25, 20.80.

MS (EI): m/z 164.20.

### Example 6

Synthesis of methyl methyl 4-(benzyloxy)benzoate (5e):

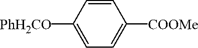

The compound of formula (5d) was synthesised following the procedure described above under Example 2 and the general procedure involving corresponding reactants. The crude material was dried under reduced pressure to provide 5e as a white solid (218 mg, 90%); The spectra data matched with values reported in the literature (Organic Letters2019, 21, 5331).

¹H NMR (400 MHz, CDCl₃) δ 8.13 - 7.89 (m, 2H), 7.57 - 7.27 (m, 5H), 7.04 - 6.95 (m, 2H), 5.12 (s, 2H), 3.88 (s, 3H).

¹³C NMR (101 MHz, CDCl₃) δ 166.85, 162.51, 136.28, 131.64, 128.71, 128.24, 127.52, 122.87, 114.49, 70.13, 51.91.

MS (EI): m/z 242.8.

### Example7

Synthesis of methyl 3-bromo-4-methylbenzoate (5f):

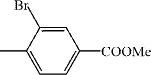

The compound of formula (5f) was synthesised following the procedure described above under Example 2 and the general procedure involving corresponding reactants. The crude material was dried under reduced pressure to provide 5f as a white solid (194 mg, 85%); The spectra data matched with values reported in the literature (Organic Letters2020, 22, 1624).

¹H NMR (400 MHz, CDCl₃) δ 8.20 (s, 1H), 7.87 (d, J= 7.9 Hz, 1H), 7.30 (d, J= 7.8 Hz, 1H), 3.91 (s, 3H), 2.45 (s, 3H).

¹³C NMR (101 MHz, CDCl₃) δ 166.85, 162.51, 136.28, 131.64, 128.71, 128.24, 127.52, 122.87, 114.49, 70.13, 51.91.

MS (EI): m/z 229.07.

### Example 8

Synthesis of 5-phenyl-1H-pyrazole (7a)

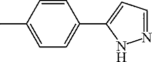

To an oven-dried 50 mL test tube equipped with a teflon coated magnetic stir bar, 1-ethynyl-4-methylbenzene (116 mg, 1 mmol) was added. Then DEE (10 mL) was added using a syringe. Then the tube was sealed by septa and an additional nitrogen balloon was placed over the tube. Next, the diazomethane solution was added through the above designed diazo-pen for 21 min. (equivalent to 1 mmol of diazomethane). After diazo exposure for 21 min, the reaction mixture was further stirred for 12 h to complete the reaction. Next reaction mixture was quenched and washed with brine(3x20 mL), then with NH₄Cl (30 mL). The organic phase was dried over Na₂SO₄ and concentrated under reduced pressureNH₄Cl (30 mL). The organic phase was dried over Na₂SO₄ and concentrated under reduced pressure to provide a white solid (65 mg, 41%). The spectra data matched with values reported in the literature (Chemical Communications2019, 55, 7986).

¹H NMR (400 MHz, CDCl₃)δ 7.48 (s, 1H), 6.84 (d, J= 8.0 Hz, 3H), 6.38 (d, J= 7.9 Hz, 2H), 5.81 (d, J= 2.1 Hz, 2H), 1.48 (s, 3H).

¹³C NMR (101 MHz, CDCl₃)δ 141.85, 134.46, 130.27, 106.83, 84.41,26.03.

MS: m/z 158.34.

### Example 9

Synthesis of 1-bromo-4-methoxybenzene (9b)

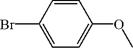

To an oven-dried 50 mL test tube equipped with a teflon coated magnetic stir bar, 4-bromo phenol (171 mg, 1 mmol) in DEE (10 mL) was added using a syringe. Then the tube was sealed by septa and an additional nitrogen balloon was placed over the tube. Next, the diazomethane solution was added through the above designed diazo-pen for 21 min. (equivalent to 1 mmol of diazomethane). After diazo exposure for 21 min, the reaction mixture was further stirred for 3 hours to complete the reaction. Next reaction mixture was quenched and washed with NaHCO₃ (3×20 mL), then with brine (30 mL). The organic phase was dried over Na₂SO₄ and concentrated under reduced pressureto give 9b as a colorless liquid (67.3 mg, 36%). The spectra data matched with values reported in the literature (AngewandteChemie International Edition2018, 57, 12869).

¹H NMR (400 MHz, CDCl₃) δ 7.38 (d, J= 9.0 Hz, 2H), 6.80 - 6.76 (m, 2H), 3.78 (s, 3H).

¹³C NMR (101 MHz, CDCl₃) δ 158.71, 132.26, 115.74, 112.84, 55.46.

MS (EI): m/z 186.04.

### Example 10

Synthesis of 4-bromo-1,2-dimethoxybenzene (9b)

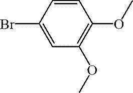

The compound of formula (9b) was synthesised following the procedure described above under Example 9 and the general procedure involving corresponding reactants. The crude material was dried under reduced pressure to provide 9b as areddish brown liquid (145.4 mg, 67%); The spectra data matched with values reported in the literature (AngewandteChemie International Edition 2018, 57, 12869).

¹H NMR (400 MHz, CDCl₃) δ 7.03 (dd, J= 8.5, 2.3 Hz, 1H), 6.98 (d, J= 2.2 Hz, 1H), 6.74 (d, J= 8.6 Hz, 1H), 3.87 (s, 3H), 3.86 (s, 3H).

¹³C NMR (101 MHz, CDCl₃) δ 149.76, 148.35, 123.40, 114.80, 112.74, 112.50, 56.11, 56.06.

MS (EI): m/z 217.06.

### Example 11

Synthesis of 3-(benzylamino)-1-diazo-4-phenylbutan-2-one (11a)

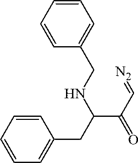

To an oven-dried 500 mL round bottom (RB) flask equipped with a teflon coated magnetic stir bar, (R)-2-(((benzyloxy)carbonyl)amino)-3-phenylpropanoic (ethyl carbonic) anhydride (1.5 g, 4 mmol) was added. Then DEE (50 ml) was added using a syringe. Then the RB was sealed by septa and an additional nitrogen balloon was placed over the flask. Next, the diazomethane solution was added through the designed diazo-pen for 126 min. (equivalent to 6 mmol of diazomethane). After diazo exposure for 126 min, the reaction mixture was further stirred for 6 h to complete the reaction. Next reaction mixture was quenched and washed with NaHCO₃ (3×20 mL), then with brine (30 mL). The organic phase was dried over Na₂SO₄ and concentrated under reduced pressure to provide 11a as a white solid (1.0 g, 79%). The spectra data matched with values reported in the literature (RSC Advances2014, 4, 37419).

¹H NMR (500 MHz, CDCl₃) δ 7.39 - 7.22 (m, 8H), 7.17 (d, J = 7.3 Hz, 2H), 5.36 (s, 1H), 5.20 (s, 1H), 5.13 - 5.01 (m, 2H), 4.48 (d, J= 5.5 Hz, 1H), 3.04 (d, J= 6.6 Hz, 2H).

¹³C NMR (101 MHz, CDCl₃) δ 192.75 (s), 155.76 (s), 136.11 (d, J= 15.3 Hz), 129.37 (s), 128.67 (d, J= 15.3 Hz), 128.19 (d, J= 17.4 Hz), 127.15 (s), 67.09 (s), 58.90 (s), 54.67 (s), 38.55 (s).

MS (EI): m/z 323.35.

### Example 12

### Synthesis of HKUST-10 M (13a)

To an oven-dried 50 mL test-tube equipped with a teflon coated magnetic stir bar, HKUST (100 mg) and DEE (10 mL) was added. Then the tube was sealed by septa and an additional nitrogen balloon was placed over the tube. Next, the diazomethane solution was added through the designed diazo-pen for 10 min. (equivalent to 0.48 mmol of diazomethane). After diazo exposure for 10 min, reaction mixture was further stirred for 2 min to complete the reaction. Next reaction MOF mixture was dried under reduced pressure to provide a formula13a.

### Example 13

### Synthesis of HKUST-20 M (13b):

To an oven-dried 50 mL test tube equipped with a teflon coated magnetic stir bar, HKUST (100 mg) and DEE (10 ml) were added. Then the tube was sealed by septa and an additional nitrogen balloon was placed over the tube. Next, the diazomethane solution was added through the designed diazo-pen for 20 min. (equivalent to 0.95 mmol of diazomethane). After diazo exposure for 20 min, the reaction mixture was further stirred for 5 min to complete the reaction. Next, the MOF mixture was dried under reduced pressure to provide a formula13b.

### Example 14

### Synthesis of HKUST-30 M (13c)

To an oven-dried 50 mL test tube equipped with a teflon coated magnetic stir bar, HKUST (100 mg) andDEE (10 ml) were added. Then the tube was sealed by septa and an additional nitrogen balloon was placed over the tube. Next, the diazomethane solution was added through the designed diazo-pen for 30 min. (equivalent to 1.42 mmol of diazomethane). After diazo exposure for 30 min, the reaction mixture was further stirred for 5 min to complete the reaction. Next,the MOF mixture was dried under reduced pressure to provide a formula 13c.

### Example 15

### Synthesis of HKUST-35 M (13d)

To an oven-dried 50 mL test tube equipped with a teflon coated magnetic stir bar, HKUST (100 mg) and DEE (10 ml) were added. Then the tube was sealed by septa and an additional nitrogen balloon was placed over the tube. Next, the diazomethane solution was added through the designed diazo-pen for 35 min. (equivalent to 1.64 mmol of diazomethane). After diazo exposure for 35 min, the reaction mixture was further stirred for an additional 5 min to complete the reaction. Next,the MOF mixture was dried under reduced pressure to provide a formula13d.

### Example 16

### Synthesis of HKUST-40 M (13e)

To an oven-dried 50 mL test tube equipped with a teflon coated magnetic stir bar, HKUST (100 mg) and DEE (10 ml) were added. Then the tube was sealed by septa and an additional nitrogen balloon was placed over the tube. Next, the diazomethane solution was added through the designed diazo-pen for 40 min. (equivalent to 1.9 mmol of diazomethane). After diazo exposure for 40 min, the reaction mixture was further stirred for an additional 5 min to complete the reaction. Next, the MOF mixture was dried under reduced pressure to provide a formula13e.

### Example 17

### Synthesis of HKUST-50 M (13f)

To an oven-dried 50 mL test tube equipped with a teflon coated magnetic stir bar, HKUST (100 mg) and DEE (10 ml) were added. Then the tube was sealed by septa and an additional nitrogen balloon was placed over the tube. Next, the diazomethane solution was added through the designed diazo-pen for 50 min. (equivalent to 2.35 mmol of diazomethane). After diazo exposure for 50 min, the reaction mixture was further stirred for an additional 5 min to complete the reaction. Next, the MOF mixture was dried under reduced pressure to provide a formula13f.

### Example 18

### Synthesis of HKUST-60 M (13g)

To an oven-dried 50 mL test tube equipped with a teflon coated magnetic stir bar, HKUST (100 mg) and DEE (10 ml) were added. Then the tube was sealed by septa and an additional nitrogen balloon was placed over the tube. Next, the diazomethane solution was added through the designed diazo-pen for 60 min. (equivalent to 2.82 mmol of diazomethane). After diazo exposure for 60 min, the reaction mixture was further stirred for an additional 5 min to complete the reaction. Next, the MOF mixture was dried under reduced pressure to provide a formula13g.

### Example 19

### Synthesis of Cotton-Fiber HKUST-60 M (13h)

To an oven-dried 50 mL test tube equipped with a teflon coated magnetic stir bar,branchedpoly(ethylenimine)(PEI) (200 mg in 50% water, mw = 25000) was dissolved in 50 mL methanol and stirred for 10 min. to get a clear suspension. Further, HKUST (500 mg) was added into the PEI solution and stirred for 10 h to get a uniform suspension. Next, prior dried cottonfibre(1.5 g) was added in MOF suspension and stirred for 3 h to get uniform HKUST MOF coating. The HKUST coated cotton fiber was further washed with methanol and dried under reduced pressure to get blue colored cottonfibre. Another side,a compound of formula (13h) was synthesised following the procedure described above under Example 18 (60 min.)with 100 mg of the cotton-fiber coated HKUST. The crude fiber was dried under reduced pressure to provide a green colored cotton fiber.

### Example 20

### Synthesis of UiO-66-60 M (13i)

The compound of formula (13i) was synthesised following the procedure described above under Example 18 and the general procedure involving corresponding UiO-66 MOF. The crude material was dried under reduced pressure to provide formula13i as a white solid.

### Example 21

### Synthesis of MIL-100 (Al)-60 M (13j)

The compound of formula (13j) was synthesised following the procedure described above under Example 18 and the general procedure involving corresponding MIL-100 (Al). The crude material was dried under reduced pressure to provide formula13jasa white solid.

### Example 22

### Synthesis of Eu-MOF- 60 M (13k)

The compound of formula (13k) was synthesised following the procedure described above under Example 18 and the general procedure involving corresponding Eu-MOF. The crude material was dried under reduced pressure to provide formula13ka white solid.

### Example 23

### Synthesis of MIL-101 (Cr)-60 M (13l)

The compound of formula (131) was synthesised following the procedure described above under Example 18 and the general procedure involving corresponding MIL-101 (Cr). The crude material was dried under reduced pressure to provide formula131a green color solid.

### Example 24

### Synthesis of MIL-101 (Fe)-60 M (13m)

The compound of formula (13 m) was synthesised following the procedure described above under Example 18 and the general procedure involving corresponding MIL-101 (Fe). The crude material was dried under reduced pressure to provide formula13m a brown color solid.

### General Procedure for the Fabrication of the Diazo Cube

1. A solution of formula2 in MeOH and Diethyl ether, separately a solution of formula3 comprising of aqueous KOH were taken in syringes and connected with a pump as described in FIG. 1.

2. The flow rate of the formula2 solution was kept varied as the flow rate of formula3, in accordance with the stoichiometry of reagent and substrates, and smoothly passed through perfluoroalkoxy (PTFE) tubing (inner diameter (id) = 800-1000 µm, length = 10-40 m, volume = 10.0-25.0 mL) for the reaction to occur.

3. A residence time of 1-10 min, 0-25° C., and pressure 0-1 bar were found to be enough for the diazomethane generation of formula1.

4. Continuous-flow separation of the aqueous and organic layers were performed through our- previously reported micro-separator. A residence time of 0-1 min, 0-1 bar pressure was found to be enough for the aqueous waste removal of the crude organic solution of formula1.

5. Next a solution of formula1 in DEE, separately from a solution of formula4 in DEE was taken in a bottle and connected with a pump as described in FIG. 1.

6. The flow rate of the formulal solution was kept varied as the flow rate of formula4, in accordance with the stoichiometry of reagent and substrates and smoothly passed through perfluoroalkoxy (PFA) tubing (inner diameter (id) = 800-1000 µm, length = 10-20 m, volume = 15.0-20 mL) for the reaction to occur.

7. A residence time of 0-1 min, 0-30° C., and pressure 0-1 bar was found to be enough for the esterification of formula4 to form the compound of formula5.

8. Next the removal of the excess diazomethane; the out-flowing reaction mixture was passed through the HKUST MOF filled catalyst cartridge. A residence time of 0-5 min, 0-30° C. was found to be enough for the diazomethane removal.

9. Next the reaction mixture solvent was removed under the vacuum to give the formula5.

### Example 25

### Diazo-Cube

A solution of formula2 in MeOH:DEE (1:2 ratio, 0.162 M) and a solution of KOH in water (30 wt%) were introduced into the capillary microreactor with a T-mixer using pumps. The flow rate of the formula2 solution (3 ml/min) was kept at same the rate asthe KOH solution (3 ml/min), in accordance with the stoichiometry of the reagent and substrates. The two solutions were introduced to a T-mixer in a flow rate with the ratio of 1:33 (formula2: KOH) to maintain the stoichiometry, and then passed through a PTFE tubing (id = 1000 µm, 1= 25.5 m, vol. = 20 ml) for the diazomethane generation during 3.3 min of residence time and room temperature. After the successful completion the aqueous and DEE continuous flow droplets were separated through our partially modified micro-separator (Organic Synthesis and Process Chemistry 2019, 23, 9, 1892-1899). A residence time of 1.16 min, 0-1 bar pressure was found to be enough for the aqueous waste removal of the crude organic solution of the formula. The out-flowed DEE reaction mixture was titrated with benzoic acid under the batch process to generate the 93.5 g/day of formula5 equivalent to 3265 mL/day ethereal diazomethane solution (0.21 M). Further to make a completely safe diazo-cube system (zero exposure), a solution of formulal in DEE directly connected with the recirculatory pump, and a solution of formula4(0.21 M in DEE) were taken in the bottle and connected with the pump as described in FIG. 1. The flow rate of the formula1 solution was kept at (2 ml/min.) and of formula4 at (2 ml/min), in accordance with the stoichiometry of reagent and substrates, and smoothly passed through perfluoroalkoxy (PFA) tubing (inner diameter (id) = 1000 µm, length = 6.4 m, volume = 5 mL) with a residence time of 1.25 min, 25° C. and pressure 1 bar for the reaction to occur. Next, the excess decomposition of diazomethane in the out-flowing reaction mixture was passed through the HKUST-MOF filled catalyst cartridge. Out-flowing reaction mixture solvent was removed under the vacuum to give the formula5 with 75%.

### ADVANTAGES OF THE INVENTION

Present disclosure relates to the development of an integrated continuous flow multi-operational protocol system for the synthesis of diazomethane and thereof.
Disclosure further relates to the said process for automated production of diazomethane total process system in 4.4 min. time with improved yield.
Disclosure further relates to the said process for diazo-pen or diazo-cube applicable for selected MOF printing applications.
The additional newly invented solid quencher powder and filter research ultimately enable to provide the identification of a completely safe environment for the integrated continuous synthesis of modern small molecule pharmaceuticals, including enantiopure APIs to fill the future gap for quick manufacturing of the late stage functionalized biologically active compounds.

We claim:

1. A process for producing and quenching diazomethane of Formula 1 (CH₂N₂), with an automated apparatus, the process comprising:
i. continuously flowing a stock solution of N-methyl-N-nitroso amine of formula 2, 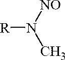
Formula 2, wherein R is an electron-withdrawing radical;
in an organic solvent and mixing with an aqueous inorganic base at a T-mixer to form a mixture and further passing the mixture through a capillary micro reactor at a temperature in a range of 20 to 30° C. to form diazomethane,
ii. separating an aqueous layer and an organic layer, wherein the organic layer comprises 0.1-0.4 M diazomethane, with a continuous flow micro-separator;
iii. reacting the organic layer with a carboxylic acid, phenol, an alkyne, an anhydride, a carboxyl metal organic framework (MOF), or MOF coated cotton to form a corresponding ester, a pyrazole, an ether, a diazo ketone, a stable carboxyl MOF or a stable MOF coated cotton fiber.

2. The process as claimed in claim 1, wherein a concentration of the diazomethane in the organic layer is maintained at about 0.1-0.4 M.

3. The process as claimed in claim 1, wherein R is a radical the portion of which that is bonded to the amine nitrogen atom shown in said formula is a member selected from the group consisting of —SO—, —C(═O)—, and —C(═NH)—.

4. The process as claimed in claim 1, wherein the N-methyl-N-nitroso amine is selected from the group consisting of N-methyl-N′-nitro-N-nitrosoguanidine, N-methyl-N-nitrosourea, N-methyl-N-nitrosocarbamate, N-methyl-N-nitrosourethane and N-methyl-N-nitroso-p-toluenesulfonamide.

5. The process as claimed in claim 1, wherein the inorganic base is potassium hydroxide.

6. The process as claimed in claim 1, wherein the capillary micro-reactor comprises a material selected from the group consisting of perfluoroalkoxy alkane (PFA), polytetrafluoroethylene (PTFE), and polyethylene (PE).

7. The process as claimed in claim 1, wherein the capillary micro-reactor has an inner diameter of at least about 1 mm and an outer diameter of 1/16 inches.

8. The process as claimed in claim 1, wherein the organic solvent is an ether, or methanol.

9. The process as claimed in claim 1, wherein the organic solvent is diethyl ether, methanol and said temperature maintained in said reaction vessel of room temperature (20-30° C.).

10. The process as claimed in claim 1, wherein the micro-separator is a hydrophobic based membrane separator.

11. The process as claimed in claim 1, wherein the ester is selected from the group consisting of methyl benzoate, methyl 4-nitrobenzoate, methyl 4-ethoxybenzoate, methyl 3,5-dimethylbenzoate, methyl 4-(benzyloxy)benzoate, and methyl 4-(benzyloxy)benzoate.

12. The process as claimed in claim 1, wherein the pyrazole is 5-(p-tolyl)-1H-pyrazole, and the diazoketone is (R)-benzyl (4-diazo-3-oxo-1-phenylbutan-2-yl)carbamate.

13. The process as claimed in claim 1, wherein the ether is 1-bromo-4-methoxybenzene or 4-bromo-1,2-dimethoxybenzene.

14. The process as claimed in claim 1, wherein the ester is selected from the group consisting of (R)-benzyl (4-diazo-3-oxo-1-phenylbutan-2-yl)carbamate.

15. The process as claimed in claim 1, wherein the carboxyl MOF is selected from the group consisting of HKUST, HKUST-coated cotton fiber, UiO-66, MIL-100 (Al), Eu-MOF, MIL-101-(Cr), wherein HKUST is a combination of copper (II) and benzene-1,3,5-tricarboxylate ligand-based MOF, UiO-66 is a combination of zirconium(IV) and terephthalate ligand-based MOF, MIL-100(Al) is a combination of aluminum(III) and benzene-1,3,5-tricarboxylateligand-based MOF, Eu-MOF is europium based MOF, MIL 101(Cr) is a combination of chromium (III) and terephthalate ligand-based MOF, MIL 101(Fe) is a combination of iron (III) and terephthalate ligand-based MOF.

16. The process as claimed in claim 1, wherein the stable carboxyl MOF is selected from the group consisting of HKUST-10 M, HKUST-20 M, HKUST-30 M, HKUST-35 M, HKUST-40 M, HKUST-50 M, HKUST-60 M, HKUST-coated cotton fiber 60 M, UiO-66-60 M, MIL-100-60 M, Eu-MOF-60 M, MIL-101 (Cr)-60 M, MIL-101 (Fe)-60 M.

17. The process as claimed in claim 1, wherein the automated apparatus is a Diazo-M-pen for laboratory scale production and utilization of diazomethane or a Diazo-M-cube for industrial scale production, utilization and quenching of diazomethane.

18. An automated apparatus for producing diazomethane of Formula 1 (CH₂N₂), the automated apparatus comprising:
a pump configured to pump a stock solution of N-methyl-N-nitroso amine in an organic solvent and an aqueous inorganic base;
a capillary micro reactor configured to form diazomethane from a reaction of the N-methyl-N-nitroso amine in the organic solvent with the aqueous inorganic base;
a continuous flow micro-separator configured to separate an aqueous layer and an organic layer, wherein the organic layer comprises 0.1-0.4 M diazomethane; and
a solid MOF quencher configured to degrade unused diazomethane.

19. The automated apparatus as claimed in claim 18, wherein the continuous flow micro-separator comprises a long-serpentine tunnel sandwiched in a polytetrafluoroethylene (PTFE)-hydrophobic membrane with three alternate (PTFE) sheets with an identical dimension of groove channels sandwiched between two metal holders tightly pressed by a screw.

20. The automated apparatus as claimed in claim 18, wherein the continuous flow micro-separator has a residence time of 0-10 min and a pressure of 0-10 bar.
